SEQUENCE LISTING (6 sequences)

SEQ ID NO: 1 - protein, 299 aa, Homo sapiens
MLESHLIIYFLLAVIQFLLGIFTNGIIVVVNGIDLIKHRKMAPLDLLLSCLAVSRIFLQLFIFYVNVIVIFFIEFIMCSANCAILLFINELELWLATWLGVFYCAKVASVRHPLFIWLKMRISKLVPWMILGSLLYVSMICVFHSKYAGFMVPYFLRKFFSQNATIQKEDTLAIQIFSFVAEFSVPLLIFLFAVLLLIFSLGRHTRQMRNTVAGSRVPGRGAPISALLSILSFLILYFSHCMIKVFLSSLKFHIRRFIFLFFILVIGIYPSGHSLILILGNPKLKQNAKKFLLHSKCCQ

SEQ ID NO: 2 - protein, 309 aa, Homo sapiens
MFSPADNIFIILITGEFILGILGNGYIALVNWIDWIKKKKISTVDYILTNLVIARICLISVMVVNGIVIVLNPDVYTKNKQQIVIFTFWTFANYLNMWITTCLNVFYFLKIASSSHPLFLWLKWKIDMVVHWILLGCFAISLLVSLIAAIVLSCDYRFHAIAKHKRNITEMFHVSKIPYFEPLTLFNLFAIVPFIVSLISFFLLVRSLWRHTKQIKLYATGSRDPSTEVHVRAIKTMTSFIFFFFLYYISSILMTFSYLMTKYKLAVEFGEIAAILYPLGHSLILIVLNNKLRQTFVRMLTCRKIACMI

SEQ ID NO: 3 - protein, 307 aa, Homo sapiens
MLRVVEGIFIFVVVSESVFGVLGNGFIGLVNCIDCAKNKLSTIGFILTGLAISRIFLIWIIITDGFIQIFSPNIYASGNLIEYISYFWVIGNQSSMWFATSLSIFYFLKIANFSNYIFLWLKSRTNMVLPFMIVFLLISSLLNFAYIAKILNDYKMKNDTVWDLNMYKSEYFIKQILLNLGVIFFFTLSLITCIFLIISLWRHNRQMQSNVTGLRDSNTEAHVKAMKVLISFIILFILYFIGMAIEISCFTVRENKLLLMFGMTTTAIYPWGHSFILILGNSKLKQASLRVLQQLKCCEKRKNLRVT

SEQ ID NO: 4 - protein, 317 aa, Homo sapiens
MGGVIKSIFTFVLIVEFIIGNLGNSFIALVNCIDWVKGRKISSVDRILTALAISRISLVWLIFGSWCVSVFFPALFATEKMFRMLTNIWTVINHFSVWLATGLGTFYFLKIANFSNSIFLYLKWRVKKVVLVLLLVTSVFLFLNIALINIHINASINGYRRNKTCSSDSSNFTRFSSLIVLTSTVFIFIPFTLSLAMFLLLIFSMWKHRKKMQHTVKISGDASTKAHRGVKSVITFFLLYAIFSLSFFISVWTSERLEENLIILSQVMGMAYPSCHSCVLILGNKKLRQASLSVLLWLRYMFKDGEPSGHKEFRESS

SEQ ID NO: 5 - protein, 321 aa, Homo sapiens
MTKLCDPAESELSPFLITLILAVLLAEYLIGIIANGFIMAIHAAEWVQNKAVSTSGRILVFLSVSRIALQSLMMLEITISSTSLSFYSEDAVYYAFKISFIFLNFCSLWFAAWLSFFYFVKIANFSYPLFLKLRWRITGLIPWLLWLSVFISFSHSMFCINICTVYCNNSFPIHSSNSTKKTYLSEINVVGLAFFFNLGIVTPLIMFILTATLLILSLKRHTLHMGSNATGSNDPSMEAHMGAIKAISYFLILYIFNAVALFIYLSNMFDINSLWNNLCQIIMAAYPASHSILLIQDNPGLRRAWKRLQLRLHLYPKEWTL

SEQ ID NO: 6 - protein, 309 aa, Homo sapiens
MITFLPIIFSILIVVTFVIGNFANGFIALVNSIEWFKRQKISFADQILTALAVSRVGLLWVLVLNWYATELNPAFNSIEVRITAYNVWAVINHFSNWLATSLSIFYLLKIANFSNLIFLHLKRRVKSVVLVILLGPLLFLVCHLFVINMNQIIWTKEYEGNMTWKIKLRSAMYLSNTTVTILANLVPFTLTLISFLLLICSLCKHLKKMQLHGKGSQDPSMKVHIKALQTVTSFLLLCAIYFLSIIMSVWSFESLENKPVFMFCEAIAFSYPSTHPFILIWGNKKLKQTFLSVLWHVRYWVKGEKPSSS

(19) United States
(12) Patent Application Publication
(10) Pub. No.: US 2025/0271426 A1
(43) Pub. Date: Aug. 28, 2025

(54) REDUCTION OF BITTER TASTE OF ACTIVE PHARMACEUTICAL INGREDIENTS AND RELATED ASSAYS AND SCREENING METHODS

(71) Applicant: Firmenich Incorporated, Plainsboro, NJ (US)

(72) Inventors: Guy Servant, San Diego, CA (US); Mark Williams, San Diego, CA (US)

(21) Appl. No.: 18/858,159

(22) PCT Filed: Apr. 18, 2023
(86) PCT No.: PCT/US2023/065901

§ 371 Date: Oct. 18, 2024

Related U.S. Application Data
(60) Provisional application No. 63/332,405, filed on Apr. 19, 2022.

(30) Foreign Application Priority Data
May 31, 2022 (EP) 22176265.1

(51) Int. Cl. G01N 33/566; A61K 47/46

(52) U.S. Cl. CPC G01N 33/566; A61K 47/46; G01N 2333/726

(57) ABSTRACT

The present disclosure generally provides methods for identifying compounds that attenuate the bitter taste of active pharmaceutical ingredients (APIs). In some aspects, the disclosure provides methods of identifying compounds that attenuate the bitter taste of APIs by determining whether the compounds modulate certain T2R bitter taste receptors. In some other aspects, the disclosure provides uses of such identified compounds to attenuate the bitter taste of an ingestible composition that comprises one or more APIs, such as liquid or solid dosage forms for oral administration. In some further aspects, the disclosure provides flavored products, such as pharmaceutical products, that incorporate such ingestible compositions.

### REFERENCE TO THE SEQUENCE LISTING

The present application is being filed along with a Sequence Listing in electronic format. The Sequence Listing is provided as a file entitled SNMX.073WO.xml created on Apr. 13, 2023, which is 8,088 bytes in size. The information in the electronic format of the Sequence Listing is incorporated herein by reference in its entirety.

### BACKGROUND

### Field

The present disclosure generally provides methods for identifying compounds that attenuate the bitter taste of active pharmaceutical ingredients (APIs). In some aspects, the disclosure provides methods of identifying compounds that attenuate the bitter taste of APIs by determining whether the compounds modulate certain T2R bitter taste receptors. In some other aspects, the disclosure provides uses of such identified compounds to attenuate the bitter taste of an ingestible composition that comprises one or more APIs, such as liquid or solid dosage forms for oral administration. In some further aspects, the disclosure provides flavored products, such as pharmaceutical products, that incorporate such ingestible compositions.

### Description of the Related Art

Active pharmaceutical ingredients (APIs) are widely used today in both developed and developing nations. They are included in various over-the-counter products, as well as in prescription drug products. When feasible, it is desirable to package APIs into dosage forms designed for oral administration, as most consumers find this to be the most convenient method of administration. But many APIs impart a noticeable bitter taste. In some cases, the bitterness is so unpleasant to certain consumers that the oral administration of these APIs becomes difficult or impossible. In certain instances, the bitter APIs can be encapsulated or coated to reduce physical contact of the API with the taste receptors on the tongue and in the mouth. But this does not work for all APIs, and can add unnecessary costs and complications to the manufacture of drugs.

In humans, bitter taste is mediated by a family of 25 G protein-coupled receptors (GPCRs) that are expressed at the surface of taste receptor cells. Bitter-tasting substances bind only to a few or sometimes several of these 25 receptors. Therefore, blocking bitterness at the receptor level can be quite complicated. But when bitter blocking can be carried out successfully, such blocking can be an effective way of selectively blocking the bitter taste receptors that are implicated with a certain kind of bitterness.

Therefore, there is a need to develop strategies for identifying which bitter receptors are responsible for the bitterness of APIs and to discover compounds that can block such bitter receptors, and thereby allow for simpler oral formulations.

### SUMMARY

The present disclosure relates to the discovery that certain of the 25 T2R taste receptors are principally responsible for the human perception of the bitter taste of APIs.

Thus, the methods disclosed herein provide a means of screening for and identifying compounds that can selectively block one or more of these certain T2R receptors. The disclosure also describes the use of such compounds in combination with pharmaceutical products that contain APIs.

In a first aspect, the disclosure provides methods of identifying compounds that reduce a bitter taste of an active pharmaceutical ingredient, the methods comprising: (a) introducing a test compound to a taste receptor, which comprises a polypeptide sequence of SEQ ID NO: 1, SEQ ID NO: 2, SEQ ID NO: 3, SEQ ID NO: 4, SEQ ID NO: 5, SEQ ID NO: 6, a functional fragment of any of the foregoing, or a polypeptide sequence whose sequence is at least 90% equivalent to any of the foregoing; and (b) measuring a response of the taste receptor to the test compound by comparing an activity of the taste receptor in a presence and an absence of the test compound. In some embodiments, the methods further comprise: (c) identifying an active test compound that reduces the bitter taste of the active pharmaceutical ingredient based on the measured response; and, optionally, (d) selecting the active test compound as a compound that reduces the bitter taste of the active pharmaceutical ingredient.

In a second aspect, the disclosure provides methods of identifying compounds that reduce a bitter taste of an active pharmaceutical ingredient, the methods comprising: (a) introducing a test compound to a taste receptor in the presence of an active pharmaceutical ingredient, wherein the taste receptor comprises a polypeptide sequence of SEQ ID NO: 1, SEQ ID NO: 2, SEQ ID NO: 3, SEQ ID NO: 4, SEQ ID NO: 5, SEQ ID NO: 6, a functional fragment of any of the foregoing, or a polypeptide sequence whose sequence is at least 90% equivalent to any of the foregoing; and (b) measuring a response of the taste receptor to the test compound by comparing an activity of the taste receptor in a presence and an absence of the test compound. In some embodiments, the methods further comprise: (c) identifying an active test compound that reduces the bitter taste of the active pharmaceutical ingredient based on the measured response; and, optionally, (d) selecting the active test compound as a compound that reduces the bitter taste of an active pharmaceutical ingredient.

In a third aspect, the disclosure provides uses of an identified or selected active test compound of any embodiments of the first or second aspect to reduce a bitter taste of active pharmaceutical ingredient in an ingestible composition.

In a fourth aspect, the disclosure provides methods of reducing a bitter taste of an active pharmaceutical ingredient in an ingestible composition, the method comprising introducing to an ingestible composition comprising an active pharmaceutical ingredient and an identified or selected active test compound of any embodiments of the first or second aspect.

In a fifth aspect, the disclosure provides ingestible compositions comprising an active pharmaceutical ingredient and an identified or selected active test compound of any embodiments of the first or second aspect.

In a sixth aspect, the disclosure provides pharmaceutical products comprising an ingestible composition of the fifth aspect. In some embodiments, the pharmaceutical products are dosage forms designed for oral administration, including solid-state forms, such as tablets, powders, and the like, and liquid forms, such as solutions or suspensions.

Further aspects, and embodiments thereof, are set forth below in the Detailed Description, the Drawings, the Abstract, and the Claims.

### DETAILED DESCRIPTION

The following Detailed Description sets forth various aspects and embodiments provided herein. The description is to be read from the perspective of the person of ordinary skill in the relevant art. Therefore, information that is well known to such ordinarily skilled artisans is not necessarily included.

### Definitions

The following terms and phrases have the meanings indicated below, unless otherwise provided herein. This disclosure may employ other terms and phrases not expressly defined herein. Such other terms and phrases have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

A “bitter compound” or a “bitter tastant” refers to a compound that elicits a detectable bitter taste sensation in a subject or that agonizes one or more T2R bitter taste receptors. A “bitter blocker” refers to a compound that inhibits the binding of a bitter compound to one or more T2R bitter taste receptors. In the context of this disclosure, the terms “inhibit” or “block” do not necessarily require complete inhibition or blocking, but encompass any reduction in the binding activity of the bitter compound.

An “active pharmaceutical ingredient” refers to the biologically active ingredient in a drug product. This includes substances that are sold over the counter without a prescription, as well as substances for which a prescription is required.

A “functional fragment” refers to a portion of a polypeptide sequence to which the umami compound or the test compound binds. Polypeptide sequences often contain certain amino acids that do not actively participate in binding, but which may serve other purposes.

In some instances, these non-functioning parts of the polypeptide sequence can be removed or partially replaced, while leaving the functional portion of the sequence intact. These modified proteins are said to comprise a functional fragment of the original polypeptide sequence.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure, and are not meant to be limiting in any fashion. Nor do these phrases indicate any kind of preference for the disclosed embodiment.

As used herein, “comprise” or “comprises” or “comprising” or “comprised of” refer to groups that are open, meaning that the group can include additional members in addition to those expressly recited. For example, the phrase, “comprises A” means that A must be present, but that other members can be present too. The terms “include,” “have,” and “composed of” and their grammatical variants have the same meaning. In contrast, “consist of” or “consists of” or “consisting of” refer to groups that are closed. For example, the phrase “consists of A” means that A and only A is present.

As used herein, “optionally” means that the subsequently described event(s) may or may not occur. In some embodiments, the optional event does not occur. In some other embodiments, the optional event does occur one or more times.

As used herein, “or” is to be given its broadest reasonable interpretation, and is not to be limited to an either/or construction. Thus, the phrase “comprising A or B” means that A can be present and not B, or that B is present and not A, or that A and B are both present. Further, if A, for example, defines a class that can have multiple members, e.g., A₁ and A₂, then one or more members of the class can be present concurrently.

Other terms are defined in other portions of this description, even though not included in this subsection.

### Polypeptide Sequences of Taste Receptors

The present disclosure provides certain polypeptide sequences that are useful in the simulating in vitro the response that certain human taste receptors would exhibit towards such compounds when ingested orally.

In some embodiments, the disclosure provides a polypeptide sequence of SEQ ID NO: 1, or a functional fragment thereof, or a polypeptide sequence whose sequence is at least 90% equivalent, or at least 95% equivalent, or at least 97% equivalent, to either of the foregoing. For purposes of clarity and to avoid any confusion, the polypeptide sequence corresponding to SEQ ID NO: 1, starting with its N-terminus, is: MLESHLIIYFLLAV IQFLLGIFTNGIIVVVNGIDLIKHRKMAPLDLLLSCLAVSRIFLQLFTFYVNVIVIFFIEFI MCSANCAILLFINELELWLATWLGVFYCAKVASVRHPLFIWLKMRISKLVPWMILGS LLYVSMICVFHSKYAGFMVPYFLRKFFSQNATIQKEDTLAIQIFSFVAEFSVPLLIFLFA VLLLIFSLGRHTRQMRNTVAGSRVPGRGAPISALLSILSFLILYFSHCMIKVFLSSLKFH IRRFIFLFFILVIGIYPSGHSLILILGNPKLKQNAKKFLLHSKCCQ, using the standard single-letter amino acid codes.

In some embodiments, the disclosure provides a polypeptide sequence of SEQ ID NO: 2, or a functional fragment thereof, or a polypeptide sequence whose sequence is at least 90% equivalent, or at least 95% equivalent, or at least 97% equivalent, to either of the foregoing. For purposes of clarity and to avoid any confusion, the polypeptide sequence corresponding to SEQ ID NO: 2, starting with its N-terminus, is: MFSPADNIFIILITGEFIL GILGNGYIALVNWIDWIKKKKISTVDYILTNLVIARICLISVMVVNGIVIVLNPDVYTK NKQQIVIFTFWTFANYLNMWITTCLNVFYFLKIASSSHPLFLWLKWKIDMVVHWILL GCFAISLLVSLIAAIVLSCDYRFHAIAKHKRNITEMFHVSKIPYFEPLTLFNLFAIVPFIV SLISFFLLVRSLWRHTKQIKLYATGSRDPSTEVHVRAIKTMTSFIFFFFLYYISSILMTFS YLMTKYKLAVEFGEIAAILYPLGHSLILIVLNNKLRQTFVRMLTCRKIACMI, using the standard single-letter amino acid codes.

In some other embodiments, the disclosure provides a polypeptide sequence of SEQ ID NO: 3, or a functional fragment thereof, or a polypeptide sequence whose sequence is at least 90% equivalent, or at least 95% equivalent, or at least 97% equivalent, to either of the foregoing. For purposes of clarity and to avoid any confusion, the polypeptide sequence corresponding to SEQ ID NO: 3, starting with its N-terminus, is: MLRVVEGIFIFVVVSES VFGVLGNGFIGLVNCIDCAKNKLSTIGFILTGLAISRIFLIWIIITDGFIQIFSPNIYASGNL IEYISYFWVIGNQSSMWFATSLSIFYFLKIANFSNYIFLWLKSRTNMVLPFMIVFLLISS LLNFAYIAKILNDYKMKNDTVWDLNMYKSEYFIKQILLNLGVIFFFTLSLITCIFLIISL WRHNRQMQSNVTGLRDSNTEAHVKAMKVLISFIILFILYFIGMAIEISCFTVRENKLLL MFGMTTTAIYPWGHSFILILGNSKLKQASLRVLQQLKCCEKRKNLRVT, using the standard single-letter amino acid codes.

In some other embodiments, the disclosure provides a polypeptide sequence of SEQ ID NO: 4, or a functional fragment thereof, or a polypeptide sequence whose sequence is at least 90% equivalent, or at least 95% equivalent, or at least 97% equivalent, to either of the foregoing. For purposes of clarity and to avoid any confusion, the polypeptide sequence corresponding to SEQ ID NO: 4, starting with its N-terminus, is: MGGVIKSIFTFVLIVE FIIGNLGNSFIALVNCIDWVKGRKISSVDRILTALAISRISLVWLIFGSWCVSVFFPALF ATEKMFRMLTNIWTVINHFSVWLATGLGTFYFLKIANFSNSIFLYLKWRVKKVVLVL LLVTSVFLFLNIALINIHINASINGYRRNKTCSSDSSNFTRFSSLIVLTSTVFIFIPFTLSLA MFLLLIFSMWKHRKKMQHTVKISGDASTKAHRGVKSVITFFLLYAIFSLSFFISVWTS ERLEENLIILSQVMGMAYPSCHSCVLILGNKKLRQASLSVLLWLRYMFKDGEPSGHK EFRESS, using the standard single-letter amino acid codes.

In some other embodiments, the disclosure provides a polypeptide sequence of SEQ ID NO: 5, or a functional fragment thereof, or a polypeptide sequence whose sequence is at least 90% equivalent, or at least 95% equivalent, or at least 97% equivalent, to either of the foregoing. For purposes of clarity and to avoid any confusion, the polypeptide sequence corresponding to SEQ ID NO: 5, starting with its N-terminus, is: MTKLCDPAESELSPFL ITLILAVLLAEYLIGIIANGFIMAIHAAEWVQNKAVSTSGRILVFLSVSRIALQSLMMLE ITISSTSLSFYSEDAVYYAFKISFIFLNFCSLWFAAWLSFFYFVKIANFSYPLFLKLRWR ITGLIPWLLWLSVFISFSHSMFCINICTVYCNNSFPIHSSNSTKKTYLSEINVVGLAFFFN LGIVTPLIMFILTATLLILSLKRHTLHMGSNATGSNDPSMEAHMGAIKAISYFLILYIFN AVALFIYLSNMFDINSLWNNLCQIIMAAYPASHSILLIQDNPGLRRAWKRLQLRLHLY PKEWTL, using the standard single-letter amino acid codes.

In some other embodiments, the disclosure provides a polypeptide sequence of SEQ ID NO: 6, or a functional fragment thereof, or a polypeptide sequence whose sequence is at least 90% equivalent, or at least 95% equivalent, or at least 97% equivalent, to either of the foregoing. For purposes of clarity and to avoid any confusion, the polypeptide sequence corresponding to SEQ ID NO: 6, starting with its N-terminus, is: MITFLPIIFSILIVVTFVI GNFANGFTALVNSIEWFKRQKISFADQILTALAVSRVGLLWVLVLNWYATELNPAFN SIEVRITAYNVWAVINHFSNWLATSLSIFYLLKIANFSNLIFLHLKRRVKSVVLVILLGP LLFLVCHLFVINMNQIIWTKEYEGNMTWKIKLRSAMYLSNTTVTILANLVPFTLTLIS FLLLICSLCKHLKKMQLHGKGSQDPSMKVHIKALQTVTSFLLLCAIYFLSIIMSVWSF ESLENKPVFMFCEAIAFSYPSTHPFILIWGNKKLKQTFLSVLWHVRYWVKGEKPSSS, using the standard single-letter amino acid codes.

The polypeptide sequences of the foregoing aspects and embodiments can be present in any suitable composition. In some embodiments, one or more polypeptide sequences of the foregoing aspects and embodiments is present in a non-naturally occurring composition, such as an in vitro assay. In some further such embodiments, the polypeptide sequences of the foregoing aspects and embodiments are expressed on the surface of cells, such as on the cells of a eukaryotic cell line.

### Screening Methods

In certain aspects, the disclosure provides methods of identifying compounds that reduce a bitter taste of an active pharmaceutical ingredient (API), the methods comprising: (a) introducing a test compound to a taste receptor, which comprises a polypeptide sequence of SEQ ID NO: 1, SEQ ID NO: 2, SEQ ID NO: 3, SEQ ID NO: 4, SEQ ID NO: 5, SEQ ID NO: 6, a functional fragment of any of the foregoing, or a polypeptide sequence whose sequence is at least 90% equivalent to any of the foregoing; and (b) measuring a response of the taste receptor to the test compound by comparing an activity of the taste receptor in the presence and the absence of the test compound. In some embodiments, the methods further comprise: (c) identifying an active test compound that reduces the bitter taste of the API based on the measured response; and, optionally, (d) selecting the active test compound as a compound that reduces the bitter taste of the API.

In certain aspects, the disclosure provides methods of identifying compounds that reduce a bitter taste of an active pharmaceutical ingredient (API), the methods comprising: (a) introducing a test compound to a taste receptor in the presence of an API, wherein the taste receptor comprises a polypeptide sequence of SEQ ID NO: 1, SEQ ID NO: 2, SEQ ID NO: 3, SEQ ID NO: 4, SEQ ID NO: 5, SEQ ID NO: 6, a functional fragment of any of the foregoing, or a polypeptide sequence whose sequence is at least 90% equivalent to any of the foregoing; and (b) measuring a response of the taste receptor to the test compound by comparing an activity of the taste receptor in the presence and the absence of the test compound. In some embodiments, the methods further comprise: (c) identifying an active test compound that reduces the bitter taste of the APT based on the measured response; and, optionally, (d) selecting the active test compound as a compound that reduces the bitter taste of the API.

The active pharmaceutical ingredient (API) can be any suitable such compound. For example, in some embodiments, the API is atropine, brinzolamide, chloramphenicol, chloroquine, clindamycin, dexamethasone, digoxin, diltiazem, diphenhydramine, docusate, dorzolamide, doxepin, doxylamine, enalapril, erythromycin, esomeprazole, famotidine, gabapentin, ginkgolide A, guaifenesin, L-histidine, lomefloxacin, methylprednisolone, ofloxacin, oleuropein, oxyphenonium, pirenzepine, prednisone, ranitidine, trapidil, trimethoprim, cetirizine, acetaminophen, procainamide, theophylline, amoxicillin, chloramphenicol, ciprofloxacin, clarithromycin, dapsone, enoxacin, metronidazole, sulfamethoxazole, clopidogrel, warfarin, apixaban, fluvoxamine, venlafaxine, citalopram, glimepiride, diphenidol, micronazole, colchicine, azelastine, brompheniramine, chlorpheniramine, fexofenadine, eprosartan, hydrochlorothiazide, labetalol, timolol, tolazoline, methimazole, baricitinib, clonixin, diclofenac, flufenamic acid, mefenamic acid, niflumic acid, quinacrine, quinine, caffeine, lasmitidan, haloperidol, cetylpyridinium chloride, chlorohexidine gluconate, chlorohexidine digluconate, dequalinium, oxybutynin chloride, oxyphenonium, secobarbital sodium, abamaciclib, dextromethorphan, hydrocortisone, eucalyptol, yohimbine, azathioprine, fujimycin, aloin, bisacodyl, lidocaine, pemirolast, cromolyn, carisoprodol, orphenadrine, papaverine, edrophonium, berberine, omeprazole, pantoprazole, docusate, or any combination thereof. In some other embodiments, the API is an analgesic, an antacid, an antiarrymthic agent, an antibiotic, an anticholinergic agent, an anticlotting agent, an anticoagulant, an anticonvulsant agent, an antidepressant, an antidiabetic agent, an antiemetic agent, an antifungal agent, an antiglaucoma agent, an antigout agent, an antihistamine, an antihypertensive agent, an antihyperthyroid agent, an anti-inflammatory, an antimalarial agent, an antimigraine agent, an antipsychotic, an antiseptic, an antispasmodic agent, a barbiturate, an anticancer agent, a corticosteroid, a cough suppressant, an expectorant, a glucocorticoid, an immunosuppressive agent, a laxative, a local anesthetic, a mast cell stabilizer, a muscle relaxant, a muscle strengthener, a supplement, a proton pump inhibitor, or a stool softener.

It was discovered that the six of the taste receptors identified as SEQ ID NOS. 1-6 are each implicated in some way in connection with the human perception of the bitter taste of certain APIs. Therefore, in the introduction step (a) of either of the foregoing aspects, the taste receptor can comprise any combination of these polypeptide sequences. In some embodiments, the taste receptor comprises only one of the polypeptide sequences.

For example, in some embodiments, the taste receptor comprises a polypeptide sequence of SEQ ID NO: 1, a functional fragment thereof, or a polypeptide sequence whose sequence is at least 90% equivalent, or at least 95% equivalent, or at least 97% equivalent, to either of the foregoing. In some further embodiments, the taste receptor comprises: a polypeptide sequence of SEQ ID NO: 1, or a polypeptide sequence whose sequence is at least 90% equivalent, or at least 95% equivalent, or at least 97% equivalent, thereto.

For example, in some embodiments, the taste receptor comprises a polypeptide sequence of SEQ ID NO: 2, a functional fragment thereof, or a polypeptide sequence whose sequence is at least 90% equivalent, or at least 95% equivalent, or at least 97% equivalent, to either of the foregoing. In some further embodiments, the taste receptor comprises: a polypeptide sequence of SEQ ID NO: 2, or a polypeptide sequence whose sequence is at least 90% equivalent, or at least 95% equivalent, or at least 97% equivalent, thereto.

For example, in some embodiments, the taste receptor comprises a polypeptide sequence of SEQ ID NO: 3, a functional fragment thereof, or a polypeptide sequence whose sequence is at least 90% equivalent, or at least 95% equivalent, or at least 97% equivalent, to either of the foregoing. In some further embodiments, the taste receptor comprises: a polypeptide sequence of SEQ ID NO: 3, or a polypeptide sequence whose sequence is at least 90% equivalent, or at least 95% equivalent, or at least 97% equivalent, thereto.

For example, in some embodiments, the taste receptor comprises a polypeptide sequence of SEQ ID NO: 4, a functional fragment thereof, or a polypeptide sequence whose sequence is at least 90% equivalent, or at least 95% equivalent, or at least 97% equivalent, to either of the foregoing. In some further embodiments, the taste receptor comprises: a polypeptide sequence of SEQ ID NO: 4, or a polypeptide sequence whose sequence is at least 90% equivalent, or at least 95% equivalent, or at least 97% equivalent, thereto.

For example, in some embodiments, the taste receptor comprises a polypeptide sequence of SEQ ID NO: 5, a functional fragment thereof, or a polypeptide sequence whose sequence is at least 90% equivalent, or at least 95% equivalent, or at least 97% equivalent, to either of the foregoing. In some further embodiments, the taste receptor comprises: a polypeptide sequence of SEQ ID NO: 5, or a polypeptide sequence whose sequence is at least 90% equivalent, or at least 95% equivalent, or at least 97% equivalent, thereto.

For example, in some embodiments, the taste receptor comprises a polypeptide sequence of SEQ ID NO: 6, a functional fragment thereof, or a polypeptide sequence whose sequence is at least 90% equivalent, or at least 95% equivalent, or at least 97% equivalent, to either of the foregoing. In some further embodiments, the taste receptor comprises: a polypeptide sequence of SEQ ID NO: 6, or a polypeptide sequence whose sequence is at least 90% equivalent, or at least 95% equivalent, or at least 97% equivalent, thereto.

In some embodiments of any of the foregoing embodiments, the introducing step (a) comprises introducing the test compound to any combination of two, three, four, five, or six of the polypeptide sequences identified above. In certain instances, this would involve screening against the two or more polypeptide sequences in separate assays. For example, in some embodiments, the introducing step (a) comprises introducing the test compound to a polypeptide sequence of SEQ ID NO: 3 and introducing the test compound to one or more of a polypeptide sequence of SEQ ID NO: 1, a polypeptide sequence of SEQ ID NO: 2, a polypeptide sequence of SEQ ID NO: 4, a polypeptide sequence of SEQ ID NO: 5, and a polypeptide sequence of SEQ ID NO: 6. For example, in some other embodiments, the introducing step (a) comprises introducing the test compound to a polypeptide sequence of SEQ ID NO: 6 and introducing the test compound to one or more of a polypeptide sequence of SEQ ID NO: 1, a polypeptide sequence of SEQ ID NO: 2, a polypeptide sequence of SEQ ID NO: 3, a polypeptide sequence of SEQ ID NO: 4, and a polypeptide sequence of SEQ ID NO: 5. For example, in some other embodiments, the introducing step (a) comprises introducing the test compound to a polypeptide sequence of SEQ ID NO: 5 and introducing the test compound to one or more of a polypeptide sequence of SEQ ID NO: 1, a polypeptide sequence of SEQ ID NO: 2, a polypeptide sequence of SEQ ID NO: 3, a polypeptide sequence of SEQ ID NO: 4, and a polypeptide sequence of SEQ ID NO: 6.

The initial introducing step (a) can be carried out in any suitable way. For example, in some non-limiting embodiments, when introducing a test compound to taste receptor, the introducing can be carried out in a single cell-based assay. Any suitable assay can be used, according to the knowledge of those skilled in the art.

In some embodiments, it can be desirable to measure the activity of the test compound in the presence of one or more additional compounds that are known to impart a bitter taste sensation. In this way, one can determine whether the test compound enhances or blocks (antagonizes) the binding of the one or more additional compounds. In some embodiments, the one or more additional compounds comprise an API, such as any of the APIs set forth above. Thus, in some embodiments, the introducing step (a) comprises introducing the test compound to the taste receptor in the presence of an API.

The foregoing embodiments involve measuring a response of the taste receptor to the test compound. This measuring can be carried out by any suitable means. For example, in some embodiments, the taste receptor is expressed on the surface of cells, and compositions are screened against the cells expressing the taste receptor in a standard cellular assay. Measuring binding to the taste receptor can be carried out by any suitable means typically used in determining protein binding in cellular assays. Suitable methods include, but are not limited to, use of fluorescent dyes, a calcium indicator protein, a fluorescent calcium indicator, a fluorescent cAMP indicator, and the like. In some embodiments, activity of the test compound is determined by its binding to the taste receptor. In some embodiments, activity of the test compound is determined by its ability to block the binding of a bitter compound, such as a plant protein, to the taste receptor.

In some further embodiments of any of the foregoing embodiments, the methods further comprise identifying an active test compound that reduce or block bitter taste based on the measured response. According to the foregoing embodiments, the identified active test compound is one that modulates the taste receptor or antagonizes its modulation by a bitter compound, such as a plant protein.

In some embodiments of any of the foregoing embodiments, the test compound is determined to be an active compound because it modulates the taste receptor, for example, because it blocks a response of the taste receptor having an IC50 of no more than 10 mM, or no more than 1 mM or no more than 500 μM, or no more than 250 M, or no more than 100 M, or no more than 10 M, and because the test compound is not active on cells not expressing any of the polypeptide sequences of SEQ ID NOS. 1-6, or any functional fragments thereof.

In some embodiments of any of the foregoing embodiments, the test compound is determined to be an active compound because it modulates the activity of a bitter compound, such as a plant protein, to the taste receptor, for example, because it blocks a response of the taste receptor to the API with an IC50 of no more than 10 mM, or no more than, no more than 1 mM, no more than 500 M, or no more than 250 μM, or no more than 100 μM, or no more than 10 μM in the presence of a bitter compound.

In some further embodiments, the methods further comprise selecting the active test compound as a compound that reduces a bitter taste, such as a bitter taste of an API. The selecting step can be carried out by any suitable means once the active test compounds are identified.

The test compound can be any suitable compound that is amenable for use in cellular screening assays. For example, in some embodiments, the test compound is an organic compound. In some embodiments, the test compound is a naturally occurring compound. In some other embodiments, the test compound is a non-naturally occurring compound. In some embodiments, the test compound is a peptide, such as a polypeptide or an oligopeptide, an amino acid, an amino acid derivative, an amide, a nucleotide, an oligonucleotide. In some embodiments, the test compound is a plant or food extract, such as a plant or food extract obtained by bioassay-guided fractionation. In some embodiments, the test compound is a diterpene or a triterpene. In some embodiments, the test compound is a ribonucleotide or derivative thereof.

### Uses and Methods

In other aspects, the disclosure provides uses of any identified or selected active compounds of the foregoing aspects, including any embodiments or combination of embodiments thereof, as set forth above. In certain related aspects, the disclosure provides uses of any identified or selected active compounds of the foregoing aspects, including any embodiments or combination of embodiments thereof, as set forth above, to reduce a bitter taste of an ingestible composition, wherein the ingestible composition comprises one or more APIs.

The disclosure also provides methods that correspond to each of the foregoing uses. Thus, in certain related aspects, the disclosure provides methods of reducing a bitter taste of an ingestible composition, comprising introducing an amount (such as a bitter-reducing-effective amount) of any identified or selected active compounds of foregoing aspects, including any embodiments or combination of embodiments thereof, as set forth above, to the ingestible composition. In some embodiments, the ingestible composition comprises one or more APIs.

The foregoing uses and methods generally involve the use of the identified or selected active compounds in a composition containing one or more additional ingredients. For example, in at least one aspect, the disclosure provides compositions comprising any identified or selected active compounds of the foregoing aspects, including any embodiments or combination of embodiments thereof, as set forth above, wherein the identified or selected active compounds make up at least 10% by weight, or at least 20% by weight, or at least 30% by weight, or at least 40% by weight, or at least 50% by weight, of the compositions on a dry weight basis (e.g., based on the total weight of the composition excluding the weight of any liquid carrier). In a related aspect, the disclosure provides solid-state compositions comprising any identified or selected active compounds of the foregoing aspects, including any embodiments or combination of embodiments thereof, as set forth above, wherein the identified or selected active compounds make up at least 10% by weight, or at least 20% by weight, or at least 30% by weight, or at least 40% by weight, or at least 50% by weight, of the solid-state compositions, based on the total weight of composition. In another related aspect, the disclosure provides ingestible compositions comprising identified or selected active compounds of the foregoing aspects, including any embodiments or combination of embodiments thereof, as set forth above, wherein the concentration of the identified or selected active compounds in the ingestible compositions is no more than 2500 ppm (such as from 1 ppm to 2500 ppm), or no more than 2000 ppm (such as from 1 ppm to 2000 ppm), or no more than 1500 ppm (such as from 1 ppm to 1500 ppm), or no more than 1000 ppm (such as from 1 ppm to 1000 ppm), or no more than 750 ppm (such as from 1 ppm to 750 ppm), or no more than 500 ppm (such as from 1 ppm to 500 ppm), or no more than 400 ppm (such as from 1 ppm to 400 ppm), or no more than 300 ppm (such as from 1 ppm to 300 ppm), or no more than 200 ppm (such as from 1 ppm to 200 ppm), or no more than 150 ppm (such as from 1 ppm to 150 ppm), or no more than 100 ppm (such as from 1 ppm to 100 ppm), or no more than 50 ppm (such as from 1 ppm to 50 ppm), or no more than 30 ppm (such as from 1 ppm to 30 ppm), or no more than 20 ppm (such as from 1 ppm to 20 ppm),

In some further embodiments, ingestible compositions disclosed herein comprise the one or more bitterness blocking compounds. Such bitterness blocking compounds include, but are not limited to, naturally derived compounds, or synthetic compounds, such as any compounds set forth in PCT Publication WO 2020/033669, and in U.S. Pat. Nos. 8,076,491; 8,445,692; and 9,247,759.

### Pharmaceutical Products

In certain aspects, the disclosure provides pharmaceutical products comprising any compositions of the preceding aspects. In some embodiments, the disclosure provides pharmaceutical products comprising one or more compounds of the identified or selected compounds. Children often have much greater sensitivity to bitter tastes than adults, so the use of bitterness blockers can be especially useful in pharmaceutical compositions for pediatric administration.

In some embodiments, when administered to a patient, the identified or selected compounds are sterile. In one embodiment, water is a preferred vehicle when a compound as disclosed and described herein is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present pharmaceutical products, if desired, can also contain minor amounts of wetting or emulsifying agents, or pH buffering agents. In addition, auxiliary, stabilizing, thickening, lubricating and coloring agents may be used. In some instances, the pharmaceutical compositions are liquid solutions or suspensions, that include, among other things, flavorants, pH modulators, natural or artificial sweeteners, and the like.

Pharmaceutical products comprising a selected or identified compound as disclosed and described herein may be manufactured by means of conventional mixing, dissolving, granulating, dragee-making, levigating, emulsifying, encapsulating, entrapping or lyophilizing processes. Pharmaceutical products may be formulated in conventional manner using one or more physiologically acceptable carriers, diluents, excipients or auxiliaries, which facilitate processing of compounds of the present disclosure into preparations which can be used pharmaceutically.

In some embodiments, the pharmaceutical products can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In some embodiments, the pharmaceutically acceptable vehicle is a capsule.

Pharmaceutical products for oral delivery may be in the form of tablets, lozenges, aqueous or oily suspensions, granules, powders, emulsions, capsules, syrups, or elixirs, for example. Orally administered pharmaceutical compositions may contain one or more optionally agents, for example, sweetening agents such as fructose, aspartame or saccharin; flavoring agents such as peppermint, oil of wintergreen, or cherry coloring agents and preserving agents, to provide a pharmaceutically palatable preparation.

Moreover, where in tablet or pill form, the pharmaceutical compositions may be coated to delay disintegration and absorption in the gastrointestinal tract, thereby providing a sustained action over an extended period of time. Selectively permeable membranes surrounding an osmotically active driving compound are also suitable for orally administered compounds of the present disclosure. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, etc. Such vehicles are preferably of pharmaceutical grade.

For oral liquid preparations such as, for example, suspensions, elixirs and solutions, suitable carriers, excipients or diluents include water, saline, alkyleneglycols (e.g., propylene glycol), polyalkylene glycols (e.g., polyethylene glycol) oils, alcohols, slightly acidic buffers between pH 4 and pH 6 (e.g., acetate, citrate, ascorbate at between about 5 mM to about 50 mM) etc. Additionally, flavoring agents, preservatives, coloring agents, bile salts, acylcarnitines and the like may be added.

Liquid drug formulations suitable for use with nebulizers and liquid spray devices and EHD aerosol devices will typically include a compound of the present disclosure with a pharmaceutically acceptable vehicle. Preferably, the pharmaceutically acceptable vehicle is a liquid such as alcohol, water, polyethylene glycol or a perfluorocarbon. Optionally, another material may be added to alter the aerosol properties of the solution or suspension of compounds of the disclosure. Preferably, this material is liquid such as an alcohol, glycol, polyglycol or a fatty acid. Other methods of formulating liquid drug solutions or suspension suitable for use in aerosol devices are known to those of skill in the art.

### EXAMPLES

To further illustrate the present disclosure, the following examples are included. The examples should not, of course, be construed as specifically limiting the disclosure. Variations of these examples within the scope of the claims are within the purview of one skilled in the art and are considered to fall within the scope of the disclosure as described, and claimed herein. The reader will recognize that the skilled artisan, armed with the present disclosure, and skill in the art is able to prepare and use the disclosure without exhaustive examples.

### Example 1—Assays

Cells expressing a promiscuous G protein (e.g., G16g44) were transfected with vectors encoding 22 functional bitter receptor cDNAs, with one receptor per transfection. The cells were transfected directly in 384-well format high-density plates and incubated for 28 hours. Later, cells were loaded with the Ca-specific dye Fluo4-AM. After an incubation of 60 minutes, excess dye is washed off, and cells are stimulated with respective bitter agonists while simultaneously recording changes in intracellular fluorescence using a FLIPR instrument. Bitter agonist and responding receptors were identified.

The above protocol was carried out using 94 different active pharmaceutical ingredients (APIs) that span a wide range of different APIs having a diversity of chemical structures. A particular T2R receptor was identified as being responsive to a particular API if it triggered a cellular response of >10% over baseline. It was surprisingly discovered that six (6) of 22 functional T2R receptors generally account for the perceived bitterness of APIs by humans. These six (6) T2R receptors correspond to SEQ ID NOS: 1, 2, 3, 4, 5, and 6. As shown in Table 1, about 92% of the 94 screened APIs showed a positive response to one of these six (6) T2R receptors. Therefore, in general, it is possible to identify compounds that reduce the bitter taste of APIs by identifying compounds that block one or more of this limited subset of six (6) T2R receptors. Table 1 shows a “+” to indicate whether a particular T2R receptor showed a positive response to a particular API. The labels “S1”, “S2”, and the like, refer to SEQ ID NO: 1, SEQ ID NO: 2, and the like, respectively.

TABLE 1

| | S1 | S2 | S3 | S4 | S5 | S6 |
|---|---|---|---|---|---|---|
| Acetaminophen | | | | | + | |
| Famotidine | + | + | + | | + | + |
| Pirenzepine | | + | + | | + | + |
| Digoxin | | + | + | + | | + |
| Procaineamide | | | + | | + | + |
| Theophylline | | | | | | |
| Amoxicillin | + | | | | | |
| Chloramphenicol | + | + | + | | + | + |
| Ciprofloxacin | | | | | | + |
| Clarithromycin | + | | + | | + | |
| Clinamycin | | + | + | + | + | |
| Dapsone | | | + | | | |
| Enoxacin | | | | | | + |
| Erythromycin | | + | + | | + | + |
| Lomefloxacin | | + | | | | + |
| Metronidazole | | | | | | |
| Ofloxacin | | + | | | | + |
| Sulfamethoxazole | + | | | | | |
| Trimethoprim | | + | + | | + | + |
| Atropine | | + | + | | + | + |
| Clopidogrel | + | | + | | | |
| Warfarin | + | | | + | + | |
| Apixaban | | | + | | | |
| Gabapentin | | + | + | | | |
| Fluvoxamine | + | | | | | |
| Venlafaxine | | | + | | | + |
| Citalopram | | | + | + | + | + |
| Doxepin | + | + | + | + | + | + |
| Glimepiride | | | | + | | |
| Diphenidol | + | | + | + | + | + |
| Micronazole | | | | + | | |
| Brinzolamide | + | + | + | | | + |
| Dorzolamide | + | + | + | | | + |
| Colchicine | | | | | + | + |
| Azelastine | | | + | | | + |
| Brompheniramine | + | | + | + | + | + |
| Cetirizine | + | + | + | + | + | + |
| Chlorpheniramine | + | | + | + | + | + |
| Diphenhydramine | + | + | + | + | + | + |
| Doxylamine | + | + | + | + | + | + |
| Fexofenadine | + | | | | + | + |
| Ranitidine | | + | + | | + | + |
| Diltiazem | + | + | + | + | | + |
| Enalapril | + | + | | + | + | + |
| Eprosartan | + | | | | | |
| Hydrochlorothiazide | | | + | | | + |
| Labetalol | | | + | | + | + |
| Timolol | + | | + | | + | + |
| Tolazoline | | | + | | | + |
| Methimazole | | | | | | |
| Baricitinib | | | + | | | + |
| Clonixin | | | | + | | |
| Diclofenac | | | | + | | |
| Flufenamic Acid | | | | + | | |
| Mefenamic Acid | | | | + | | |
| Methylprednisolone | + | + | + | + | | + |
| Niflumic Acid | | | | + | | |
| Chloroquine | + | + | + | + | + | + |
| Quinacrine | | | | | | |
| Quinine | | | + | + | + | + |
| Caffeine | | | + | + | | + |
| Lasmitidan | | | + | | | + |
| Haloperidol | | | + | + | | |
| Cetylpyridinium Chloride | | | | | | |
| Chlorhexidine Digluconate | + | | | + | + | + |
| Chlorhexidine Gluconate | + | | | | + | + |
| Dequalinium | | | | | + | + |
| Oxybutynin Chloride | | | + | + | + | + |
| Oxyphenonium | + | + | + | | + | + |
| Secobarbital Sodium | | | | + | | |
| Abamaciclib | | | | | | + |
| Prednisone | + | + | + | + | | + |
| Dextromethorphan | + | | + | + | + | + |
| Eucalyptol | + | | | + | | |
| Yohimbine | + | | + | | | + |
| Guaifenesin | + | + | + | + | + | |
| Dexamethasone | + | + | + | + | | + |
| Hydrocortisone | | | | | | + |
| Azathioprine | | | + | | | + |
| Fujimycin | + | | | | | |
| Aloin | | | | | | |
| Bisacodyl | + | | | + | | + |
| Lidocaine | | | + | + | | |
| Pemirolast | | | | + | | |
| Cromolyn | | | | | | |
| Carisoprodol | | | | + | | |
| Orphenadrine | | | | | | + |
| Papaverine | | | + | + | | |
| Edrophonium | + | | | | + | + |
| Berberine | | | | | | |
| Esomeprazole | | + | + | + | | + |
| Omeprazole | | | + | + | | + |
| Pantoprazole | | | | + | | |
| Docusate | + | + | | + | | |

1. A method of identifying compounds that reduce a bitter taste of an active pharmaceutical ingredient, the method comprising:
(a) introducing a test compound to a taste receptor, which comprises a polypeptide sequence of SEQ ID NO: 1, SEQ ID NO: 2, SEQ ID NO: 3, SEQ ID NO: 4, SEQ ID NO: 5, SEQ ID NO: 6, a functional fragment of any of the foregoing, or a polypeptide sequence whose sequence is at least 90% equivalent to any of the foregoing; and
(b) measuring a response of the taste receptor to the test compound by comparing an activity of the taste receptor in the presence and the absence of the test compound.

2. A method of identifying compounds that reduce bitter taste of an active pharmaceutical ingredient, the method comprising:
(a) introducing a test compound to a taste receptor in the presence of an active pharmaceutical ingredient or any other agonists activating the receptor, wherein the taste receptor comprises a polypeptide sequence of SEQ ID NO: 1, SEQ ID NO: 2, SEQ ID NO: 3, SEQ ID NO: 4, SEQ ID NO: 5, SEQ ID NO: 6, a functional fragment of any of the foregoing, or a polypeptide sequence whose sequence is at least 90% equivalent to any of the foregoing; and
(b) measuring a response of the taste receptor to the test compound by comparing an activity of the taste receptor in the presence and the absence of the test compound.

3. The method of claim 1, further comprising: (c) identifying an active test compound that reduces the bitter taste of the active pharmaceutical ingredient based on the measured response.

4. The method of claim 3, further comprising (d) selecting the active test compound as a compound that reduces the bitter taste of the active pharmaceutical ingredient.

5. The method of claim 2, wherein the active pharmaceutical ingredient is an analgesic, an antacid, an antiarrymthic agent, an antibiotic, an anticholinergic agent, an anticlotting agent, an anticoagulant, an anticonvulsant agent, an antidepressant, an antidiabetic agent, an antiemetic agent, an antifungal agent, an antiglaucoma agent, an antigout agent, an antihistamine, an antihypertensive agent, an antihyperthyroid agent, an anti-inflammatory, an antimalarial agent, an antimigraine agent, an antipsychotic, an antiseptic, an antispasmodic agent, a barbiturate, an anticancer agent, a corticosteroid, a cough suppressant, an expectorant, a glucocorticoid, an immunosuppressive agent, a laxative, a local anesthetic, a mast cell stabilizer, a muscle relaxant, a muscle strengthener, a supplement, a proton pump inhibitor, or a stool softener.

6. The method of claim 5, wherein the active pharmaceutical ingredient is acetaminophen, abamaciclib, aloin, amoxicillin, atropine, apixaban, azathioprine, azelastine, baricitinib, berberine, bisacodyl, brinzolamide, brompheniramine, caffeine, carisoprodol, cetirizine, cetylpyridinium chloride, clonixin, chloramphenicol, chloroquine, chlorohexidine gluconate, chlorohexidine digluconate, chlorpheniramine, citalopram, ciprofloxacin, clarithromycin, clindamycin, clopidogrel, colchicine, cromolyn, dapsone, dexamethasone, dequalinium, dextromethorphan, diclofenac, digoxin, diltiazem, diphenhydramine, diphenidol, docusate, dorzolamide, doxepin, doxylamine, edrophonium, enalapril, enoxacin, eprosartan, erythromycin, esomeprazole, eucalyptol, famotidine, fexofenadine, flufenamic acid, fluvoxamine, fujimycin, gabapentin, ginkgolide A, glimepiride, guaifenesin, haloperidol, L-histidine, hydrochlorothiazide, hydrocortisone, labetalol, lasmitidan, lidocaine, lomefloxacin, mefenamic acid, methylprednisolone, methimazole, metronidazole, micronazole, niflumic acid, ofloxacin, oleuropein, omeprazole, orphenadrine, oxybutynin chloride, oxyphenonium, pantoprazole, papaverine, pemirolast, pirenzepine, prednisone, procainamide, quinine, quinacrine, ranitidine, secobarbital sodium, sulfamethoxazole, theophylline, timolol, tolazoline, trapidil, trimethoprim, venlafaxine, warfarin, yohimbine or any combination thereof.

7. The method of claim 1, wherein the test compound is a naturally occurring compound.

8. The method of claim 1, wherein the test compound is a non-naturally occurring compound.

9. A method of reducing a bitter taste of an ingestible composition, said method comprising introducing a bitter-reducing-effective amount of a taste-modifying compound to the ingestible composition, wherein the taste-modifying compound is the identified or selected compound of claim 3.

10. The method of claim 9, wherein the taste-modifying compound is a naturally occurring compound.

11. The method of claim 9, wherein the taste-modifying compound is a non-naturally occurring compound.

12. The method of claim 9, wherein the ingestible composition comprises an active pharmaceutical ingredient.

13. An ingestible composition comprising an active pharmaceutical ingredient and a taste-modifying compound, wherein the taste-modifying compound is the identified or selected compound of claim 3.

14. A pharmaceutical product, which comprises an ingestible composition of claim 13.

15. The method of claim 2, further comprising: (c) identifying an active test compound that reduces the bitter taste of the active pharmaceutical ingredient based on the measured response.

16. An ingestible composition comprising an active pharmaceutical ingredient and a taste-modifying compound, wherein the taste-modifying compound is the identified or selected compound of claim 15.

17. A pharmaceutical product, which comprises an ingestible composition of claim 16.
